(19) United States
(12) Patent Application Publication
(10) Pub. No.: US 2010/0197732 A1
(43) Pub. Date: Aug. 5, 2010

(54) Repaglinide Substantially Free of Dimer Impurity

(75) Inventors: Sonny Sebastian, Kerala (IN); Sasidhar Venkata Balla, Andhra Pradesh (IN); Ramamurthy Katikareddy, Andhra Pradesh (IN); Nitin Sharadchandra Pradhan, Maharashtra (IN); Jon Valgeirsson, Hafnarfjordur (IS)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/663,106

(22) PCT Filed: Jun. 5, 2008
(86) PCT No.: PCT/IB08/02548

§ 371 Date: Apr. 6, 2010

(30) Foreign Application Priority Data
Jun. 6, 2007 (IN) 1160/CHE/2007
Jul. 16, 2007 (IN) 1515/CHE/2007

(51) Int. Cl. A61K 31/4545; C07D 211/26; C07D 211/32; A61K 31/4453; A61P 3/10

(52) U.S. Cl. 514/316; 546/229; 546/234; 546/190; 514/331

(57) ABSTRACT

The present invention provides highly pure repaglinide substantially free of dimer impurity, and process for the preparation thereof. The present invention also relates to 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide, an impurity of repaglinide, and a process for preparing and isolating thereof. The present invention further relates to pharmaceutical compositions comprising solid particles of pure repaglinide substantially free of dimer impurity or pharmaceutically acceptable salts thereof, wherein 90 volume-percent of the particles (D₉₀) have a size of less than about 400 microns. The present invention also provides an optical resolution method of racemic 3-methyl-1-(2-piperidino-phenyl)-1-butylamine and use thereof for the preparation of repaglinide.

### CROSS-REFERENCE TO RELATED APPLICATIONS

This application claims the benefit of priority to Indian provisional application Nos. 1160/CHE/2007, filed on Jun. 6, 2007, and 1515/CHE/2007, filed on Jul. 16, 2007, which are incorporated herein by reference.

### FIELD OF THE INVENTION

The present invention provides highly pure repaglinide substantially free of dimer impurity, and process for the preparation thereof. The present invention also relates to 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide, an impurity of repaglinide, and a process for preparing and isolating thereof. The present invention further relates to pharmaceutical compositions comprising solid particles of pure repaglinide substantially free of dimer impurity or pharmaceutically acceptable salts thereof, wherein 90 volume-percent of the particles (D₉₀) have a size of less than about 400 microns. The present invention also provides an optical resolution method of racemic 3-methyl-1-(2-piperidino-phenyl)-1-butylamine and use thereof for the preparation of repaglinide.

### BACKGROUND OF THE INVENTION

U.S. Pat. No. 5,312,924 discloses a variety of phenylacetic acid benzylamide derivatives and their salts, processes for their preparation, pharmaceutical compositions comprising the derivatives, and method of use thereof. These compounds are hypoglycemic agents. Of these compounds, Repaglinide, (S)-(+)-2-ethoxy-4-[N-[1-(2-piperidinophenyl)-3-methyl-1-butyl]aminocarbonylmethyl]benzoic acid, is an oral blood glucose-lowering drug of the meglitinide class used in the management of type 2 diabetes mellitus (also known as non-insulin dependent diabetes mellitus or NIDDM).

Repaglinide lowers blood glucose levels by stimulating the release of insulin from the pancreas. This action of Repaglinide is dependent upon functioning beta (β) cells in the pancreatic islets. Insulin release is glucose-dependent and diminishes at low glucose concentrations. Repaglinide is represented by the following structural formula I:

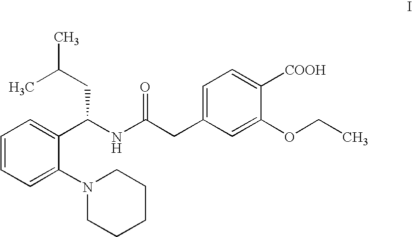

Various processes for the preparation of Repaglinide and related compounds are disclosed in U.S. Pat. No. 5,312,924, PCT Publication Nos. WO 03/027072 A1 and WO 2004/103983 A1, and U.S. Patent Application No. 2007/0123564 A1.

According to U.S. Pat. No. 5,312,924 (hereinafter referred to as the '924 patent), repaglinide is prepared by the reaction of (S)-3-methyl-1-(2-piperidinophenyl)-1-butylamine with 3-ethoxy-4-ethoxycarbonyl-phenyl acetic acid in the presence of a dehydrating agent. The dehydrating agents include ethyl chloroformate, thionyl chloride, phosphorus trichloride, phosphorus pentoxide, N,N′-dicyclohexylcarbodiimide, N,N′-dicyclohexylcarbodiimide/N-hydroxysuccinimide, N,N′-carbonyldiimidazole or N,N′-thionyldiimidazole or triphenylphosphine/carbon tetrachloride. The reaction is performed optionally in the presence of an inorganic base such as sodium carbonate or a tertiary organic base such as triethylamine, in a solvent such as methylene chloride at a temperature of −25° C. to 250° C., preferably −10° C. to the boiling temperature of the solvent used, to produce ethyl (S)-2-ethoxy-4-[N-[1-(2-piperidinophenyl)-3-methyl-1-butyl]aminocarbonylmethyl]benzoate. The subsequent removal of protecting group is preferably carried out by hydrolysis, conveniently either in the presence of an acid such as hydrochloric acid, sulphuric acid, phosphoric acid or trichloroacetic acid, or in the presence of a base such as sodium hydroxide or potassium hydroxide in a suitable solvent such as water, methanol, methanol/water, ethanol, ethanol/water, water/isopropanol or water/dioxane at −10° C. and 120° C. to produce repaglinide.

According to PCT Publication No. WO 03/027072 A1 (hereinafter referred to as the '072 application), repaglinide is prepared by the reaction of (S)-3-methyl-1-(2-piperidinophenyl)-1-butylamine with 3-ethoxy-4-ethoxycarbonyl-phenyl acetic acid in the presence of pivaloyl chloride and a base in a solvent selected from dichloromethane, toluene and xylene, to produce ethyl (S)-2-ethoxy-4-[N-[1-(2-piperidinophenyl)-3-methyl-1-butyl]aminocarbonylmethyl]benzoate followed by removal of the protecting group in presence of a base to produce repaglinide.

According to PCT Publication No. WO 2004/103983 A1 (hereinafter referred to as the '983 application), repaglinide can be prepared by the reaction of (S)-3-methyl-1-(2-piperidinophenyl)-1-butylamine with 3-ethoxy-4-ethoxycarbonyl-phenyl acetic acid in the presence of propane phosphonic acid anhydride to produce ethyl (S)-2-ethoxy-4-[N-[1-(2-piperidinophenyl)-3-methyl-1-butyl]aminocarbonylmethyl]benzoate followed by removal of the protecting group in presence of a base to produce repaglinide.

Repaglinide obtained by the processes described in the prior art mentioned above does not have satisfactory purity. Unacceptable amounts of impurities are generally formed along with repaglinide. In addition, the processes involve the additional step of column chromatographic purifications or multiple crystallizations. Methods involving column chromatographic purifications or multiple crystallizations are generally undesirable for large-scale operations, thereby making the processes commercially unfeasible.

Like any synthetic compound, repaglinide can contain extraneous compounds or impurities that can come from many sources. They can be unreacted starting materials, by-products of the reaction, products of side reactions, or degradation products. Impurities in repaglinide or any active pharmaceutical ingredient (API) are undesirable and, in extreme cases, might even be harmful to a patient being treated with a dosage form containing the API.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the International Conference on Harmonization of Technical Requirements for Registration for Human Use (“ICH”) Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the API, repaglinide, it must be analyzed for purity, typically, by HPLC, TLC or GC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. As discussed above, in the United States, the Food and Drug Administration guidelines recommend that the amounts of some impurities be limited to less than 0.1%.

Generally, impurities (side products, byproducts, and adjunct reagents) are identified spectroscopically and by other physical methods and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRt”) to identify impurities.

J. Pharm. Biomed. Anal., volume: 32(3), pages: 461-7 (2003) disclosed a list of four possible impurities of repaglinide (I, II, III and IV). These impurities were characterized as 4-carboxymethyl-2-ethoxy-benzoic acid (I), 4-cyclohexylamino-carbamoylmethyl-2-ethoxy-benzoic acid (II), 1,3-dicyclohexyl urea (III), and 1-cyclohexyl-3-[3-methyl-1-(2-piperidin-1-yl-phenyl)butyl]-urea (IV).

The present invention relates to a new impurity, whose presence was observed in repaglinide and which has not been reported in the prior art, 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide (hereinafter referred to as the ‘dimer impurity’) of formula IIa:

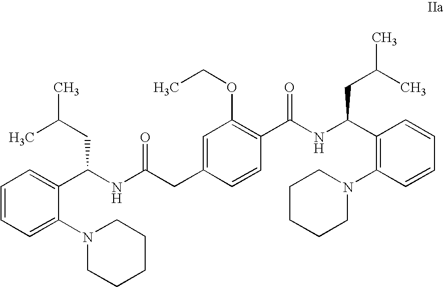

and it is identified, isolated and synthesized. The structure of the compound of formula IIa was deduced with the aid of ¹H, ¹³C NMR spectroscopy and FAB mass spectroscopy. The parent ion at 680.96 is consistent with assigned structure.

Accordingly, there remains a need for highly pure repaglinide substantially free of dimer impurity, as well as purification processes for preparing thereof.

Extensive experimentation is carried out by the present inventors to reduce the level of the dimer impurity. As a result, it has been found that the dimer impurity formed in the preparation of the repaglinide can be reduced by providing a solution of the crude repaglinide in a suitable organic solvent, contacting the solution with C₅-C₁₀ aliphatic or alicyclic hydrocarbon solvent to form a precipitate, and recovering pure repaglinide substantially free of dimer impurity. Specific organic solvents are aromatic hydrocarbons, esters, polar aprotic solvents and mixtures thereof, and more specifically aromatic hydrocarbon solvents.

Specific surface area of an active pharmaceutical ingredient may be affected by various factors. There is a general connection between Specific Surface Area and Particle Size Distribution (PSD); the smaller the Particle Size Distribution, the higher the Specific Surface Area. The rate of dissolution of a poorly-soluble drug is a rate-limiting factor in its absorption by the body. A reduction in the particle size can increase the dissolution rate of such compounds through an increase in the surface area of the solid phase that is in contact with the liquid medium, thereby resulting in an enhanced bioavailability of the compositions containing such compounds. It is generally not possible to predict the exact particle size and distribution required for any particular drug substance to achieve a specific dissolution profile or a specific in vivo behavior, as different drugs show differing dissolution characteristics with a reduction in the particle size.

Repaglinide is a white to off-white powder. The solubility of the drug substance repaglinide is quite low (9 micro gram/ml in pH 5.0 buffer solution). The lack of solubility of repaglinide creates a problem since bioavailability of a water insoluble active ingredient is usually poor. There is a need in the art to prepare active pharmaceutical ingredients such as repaglinide particles with a desired surface area to obtain formulations with greater bioavailability, and to compensate for any loss of surface area before formulation.

Hence, there is a need in the art for highly pure repaglinide substantially free of dimer impurity or a pharmaceutically acceptable salt thereof with reduced particle size distribution, which have good flow properties, and better dissolution and solubility properties to obtain formulations with greater bioavailability.

In the preparation of Repaglinide, (S)-3-methyl-1-(2-piperidinophenyl)-1-butylamine of formula III:

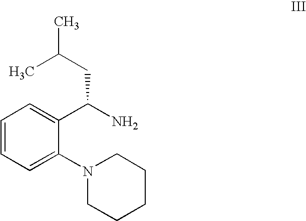

is a key intermediate. A previously known method for the synthesis of intermediate, (S)-3-methyl-1-(2-piperidinophenyl)-1-butylamine was reported in the U.S. Pat. No. 5,312,924, which involves the resolution of racemic (±)-3-methyl-1-(2-piperidino-phenyl)-1-butylamine of formula IV:

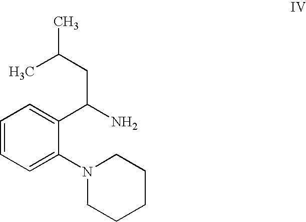

by using N-acetyl-L-glutamic acid as the optically active acid and in the presence acetone and methanol at reflux conditions and subsequent decomposition of the salt.

The (S)-amine compound of formula III obtained by the process described in the '924 patent does not have satisfactory chiral purity. The process used in the '924 patent also suffers from disadvantages such as high cost of reagent, low yields of the product, extra purification steps.

The object of the present invention is to provide a commercially useful procedure for obtaining the desired enantiomer of the compound of formula IV separately with a good yield and suitable enantiomeric purity, and its use thereof for the preparation of repaglinide. Desirable process properties include non-hazardous and environmentally friendly reagents, reduced cost, greater simplicity, increased enantiomeric and chemical purity, and increased yield of the product.

### SUMMARY OF THE INVENTION

In one aspect, the present invention provides pure repaglinide or a pharmaceutically acceptable salt thereof substantially free of dimer impurity.

In another aspect, the present invention provides repaglinide or a pharmaceutically acceptable salt thereof containing less than about 0.25% of the dimer compound II:

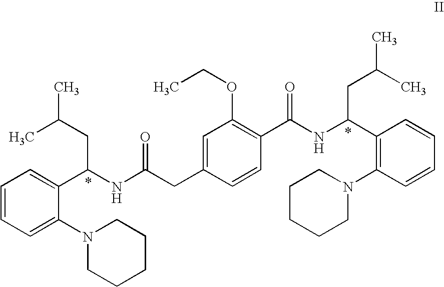

or its stereochemically isomeric forms or a mixture of stereochemically isomeric forms thereof. Preferably the repaglinide of the present invention contains less than about 0.15%, more preferably less than about 0.1%, still more preferably less than 0.05% and most preferably less than 0.02% of the dimer compound II or its stereochemically isomeric forms or a mixture of stereochemically isomeric forms thereof.

In a particular aspect, the present invention provides repaglinide or a pharmaceutically acceptable salt thereof containing less than about 0.25% of the dimer impurity of formula IIa:

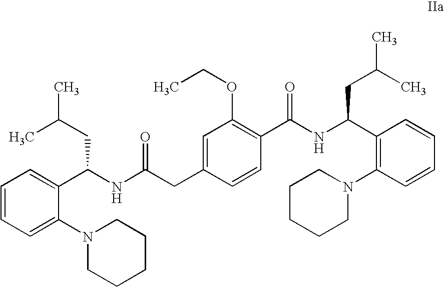

Preferably the repaglinide of the present invention contains less than about 0.15%, more preferably less than about 0.1%, still more preferably less than 0.05% and most preferably less than 0.02% of the dimer impurity.

In another aspect, the present invention provides repaglinide having total purity of greater than about 99.7%, specifically greater than about 99.9%, and more specifically greater than about 99.95% measured by HPLC.

In another aspect, the present invention encompasses a process for preparing the highly pure repaglinide substantially free of the dimer impurity or a pharmaceutically acceptable salt thereof.

In another aspect, the present invention encompasses a novel compound 2-ethoxy-N-[3-methyl-1-[2-(1-piperidinyl)phenyl]butyl]-4-[2-[[3-methyl-1-[2-(1-piperidinyl)phenyl]butyl]amino]-2-oxoethyl]benzamide, denominated dimer compound II, having the following structural formula:

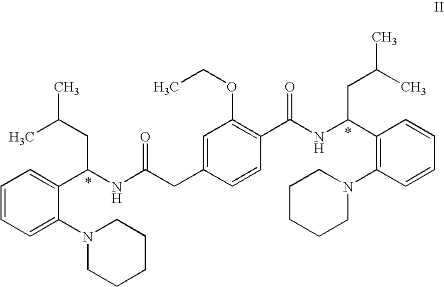

The dimer compound II contains two chiral centers (the asterisk designates the chiral centers) and therefore can exist as four optical isomers. The absolute configuration of these centers may be indicated by the stereochemical descriptors R and S, this R and S notation corresponding to the rules described in Pure Appl. Chem. 1976, 45, 11-30. Unless otherwise mentioned or indicated, the chemical designation of compounds denotes the mixture of all possible stereochemically isomeric forms, said mixtures containing all diastereomers and enantiomers of the basic molecular structure. Stereochemically isomeric forms of the compounds of formula II are obviously intended to be embraced within the scope of the invention.

In another aspect, the present invention encompasses an impurity of repaglinide 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide, denominated dimer impurity, having the following structural formula IIa:

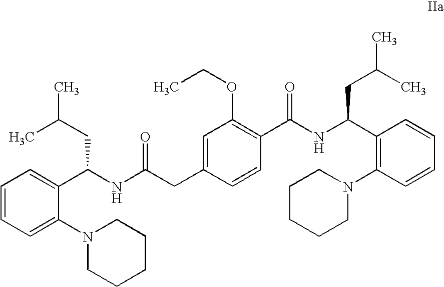

In another aspect, the present invention is directed to a process for synthesizing the dimer impurity by reaction of 3-ethoxy-4-ethoxycarbonyl phenyl acetic acid with (S)-3-methyl-1-(2-piperidinophenyl)-1-butylamine in presence of a dehydrating agent selected from boric acid or boric acid derivatives, and in a suitable solvent.

In another aspect, the present invention provides a pharmaceutical composition comprising pure repaglinide or a pharmaceutically acceptable salt thereof containing less than about 0.25% of the dimer impurity, obtained by the process of the present invention and one or more pharmaceutically acceptable excipients.

In another aspect, the present invention provides solid particles of pure repaglinide or a pharmaceutically acceptable salt thereof containing less than about 0.25% of the dimer impurity, wherein 90 volume-percent of the particles (D₉₀) have a size of less than or equal to about 400 microns, specifically less than or equal to about 300 microns, more specifically less than or equal to about 100 microns, still more specifically less than or equal to about 60 microns and most specifically less than or equal to about 15 microns.

In another aspect, the present invention provides a pharmaceutical composition comprising solid particles of pure repaglinide or a pharmaceutically acceptable salt thereof containing less than about 0.25% of the dimer impurity, wherein 90 volume-percent of the particles (D₉₀) have a size of less than or equal to about 400 microns, specifically less than or equal to about 300 microns, more specifically less than or equal to about 100 microns, still more specifically less than or equal to about 60 microns and most specifically less than or equal to about 15 microns, of the present invention and one or more pharmaceutically acceptable excipients.

In another aspect, provided herein is an efficient, convenient, commercially viable and environment friendly resolution process for the preparation of enantiomerically pure repaglinide intermediate, (S)-3-methyl-1-(2-piperidinophenyl)-1-butyl amine.

In another aspect, the present invention provides (S)-3-methyl-1-(2-piperidinophenyl)-1-butylamine having enantiomeric purity greater than about 98%, specifically greater than about 99.9%, more specifically greater than about 99.95%, and most specifically greater than about 99.98% measured by HPLC.

In still another aspect, the present invention also encompasses the use of enantiomerically pure (S)-3-methyl-1-(2-piperidinophenyl)-1-butylamine obtained by the process of present invention for preparing repaglinide.

### DETAILED DESCRIPTION OF THE INVENTION

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use. Representative alkali or alkaline earth metal salts include the sodium, calcium, potassium and magnesium salts, and the like.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, and combinations thereof.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, and combinations thereof.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, and combinations thereof.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, and combinations thereof.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, and combinations thereof.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP).

As used herein, D_{X} means that X percent of the particles have a diameter less than a specified diameter D. Thus, a D₉₀ of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

The term “crude repaglinide” as used herein refers to repaglinide which is at least 90% pure, most suitably 95% pure, and more preferably at least 99% pure.

According to one aspect of the present invention, there is provided pure repaglinide or a pharmaceutically acceptable salt thereof substantially free of dimer impurity.

As used herein, “pure repaglinide substantially free of dimer impurity” refers to repaglinide containing less than about 0.25% of the dimer compound II:

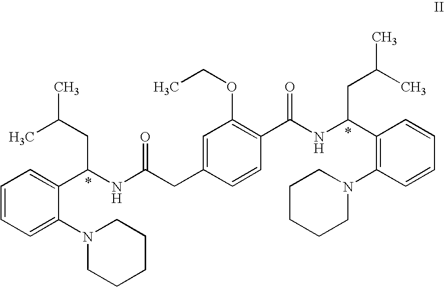

or a stereochemically isomeric form or a mixture of stereochemically isomeric forms thereof, preferably the dimer impurity of formula IIa:

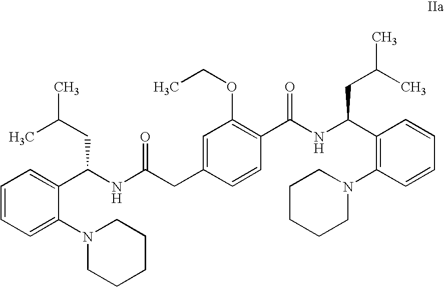

Preferably the repaglinide of the present invention contains less than about 0.15%, more preferably less than about 0.1%, still more preferably less than 0.05% and most preferably less than 0.02% of the dimer compound II, or a stereochemically isomeric form or a mixture of stereochemically isomeric forms thereof.

More preferably the repaglinide of the present invention contains less than about 0.15%, more preferably less than about 0.1%, still more preferably less than 0.05% and most preferably less than 0.02% of the dimer impurity of formula IIa. For example, the pure repaglinide of the present invention contains the dimer impurity at less than about 0.2%, preferably less than about 0.1%, more preferably less than 0.05% and most preferably less than 0.02%. The purity is preferably measured by HPLC.

The pure repaglinide of the present invention has a total purity of greater than about 99%, specifically greater than about 99.90%, and more specifically greater than about 99.95% as measured by HPLC. For example, the total purity of the pure repaglinide of the present invention can be about 99% to about 99.95%, or about 99% to about 99.99%.

According to another aspect of the present invention, there is provided a process for the purification of repaglinide, which comprises:

a) providing a solution of crude repaglinide in a suitable solvent;
b) admixing the solution of step-(a) with an anti-solvent; and
c) recovering pure repaglinide substantially free of dimer impurity.

The solvent used in step-(a) is selected from the group consisting of aromatic hydrocarbons, esters, polar aprotic solvents, and mixtures thereof. Exemplary aromatic hydrocarbon solvents include, but are not limited to, C₆ to C₁₂ aromatic hydrocarbon solvents such as benzene, alkyl substituted benzenes, and mixtures thereof. Specific aromatic hydrocarbon solvents are toluene, xylene, and mixtures thereof, and more specifically toluene. Exemplary ester solvents include, but are not limited to, C₂ to C₆ alkyl acetates such as methyl acetate, ethyl acetate, n-propyl acetate, isopropyl acetate, n-butyl acetate, isobutyl acetate, tert-butyl acetate, ethyl formate, and mixtures thereof. Specific ester solvents are ethyl acetate, isopropyl acetate, and mixtures thereof. Exemplary polar aprotic solvents include, but are not limited to, N,N-dimethylformamide, N,N-dimethylacetamide, dimethylsulfoxide, and mixtures thereof. Most preferred solvent used in step-(a) is toluene.

Step-(a) of providing a solution of crude repaglinide includes dissolving crude repaglinide in the solvent, or obtaining an existing solution from a previous processing step.

Preferably the crude repaglinide is dissolved in the solvent at a temperature of above about 25° C., more preferably at about 30° C. to about 110° C., and still more preferably at about 30° C. to about 80° C.

The solution in step-(a) may also be prepared by reacting (S)-3-methyl-1-(2-piperidinophenyl)-1-butylamine with 3-ethoxy-4-ethoxycarbonyl phenyl acetic acid in the presence of a dehydrating agent, optionally in the presence of an organic or inorganic base, in a suitable solvent under suitable conditions, followed by hydrolysis in the presence of an acid or a base to produce a reaction mass containing crude repaglinide followed by usual work up such as washings, extractions etc., and dissolving the resulting crude repaglinide in the solvent at a temperature of above about 25° C., more preferably at about 25° C. to about 110° C. and still more preferably at about 25° C. to about 80° C.

Alternatively, the solution in step-(a) may be prepared by treating an acid addition salt of repaglinide with a base to liberate repaglinide and dissolving the repaglinide in the solvent.

As acid addition salts, the salts derived from a therapeutically acceptable acid such as hydrochloric acid, acetic acid, propionic acid and, more particularly, from a di- or polybasic acid such as phosphoric acid, succinic acid, maleic acid, fumaric acid, citric acid, glutaric acid, citraconic acid, glutaconic acid, and tartaric acid can be used.

The treatment of an acid addition salt with base is carried out in any solvent and the selection of solvent is not critical. A wide variety of solvents such as chlorinated solvents, hydrocarbon solvents, ether solvents, alcoholic solvents, ketonic solvents, ester solvents etc., can be used.

The base can be inorganic or organic. Preferable base is an inorganic base selected from alkali metal hydroxides, carbonates and bicarbonates. Preferable alkali metal is sodium or potassium.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing repaglinide by removing charcoal. Preferably, finely powdered carbon is an active carbon.

The anti-solvent used in step-(b) is selected from the group consisting of C₃ to C₇ straight or cyclic aliphatic hydrocarbon solvents such as hexane, heptane, cyclopentane, cyclohexane, cycloheptane, and mixtures thereof. Specific anti-solvents are hexane, heptane, cyclohexane, and mixtures thereof, and more specifically cyclohexane.

As used herein, “anti-solvent” means a solvent which when added to an existing solution of a substance reduces the solubility of the substance.

The admixing in step-(b) may be done in any order, for example, the anti-solvent may be added to the solution, or alternatively, the solution may be added to the anti-solvent. When the hot solution is added to the anti-solvent, the temperature difference causes the fast crystallization. The addition may be carried out drop wise or in one volume. The addition is preferably carried out at a temperature of about 40° C. to about 80° C. for at least 20 minutes, and more preferably at a temperature of about 50° C. to about 75° C. from about 30 minutes to about 4 hours. After completion of the addition process, the admixture may preferably be cooled at a temperature of below 30° C., and more preferably at about 0° C. to about 30° C.

The recovery of pure repaglinide substantially free of dimer impurity in step-(c) can be performed by filtration or centrifugation.

If required, pure repaglinide substantially free of dimer impurity obtained in step-(c) may be converted into pharmaceutically acceptable salts by conventional methods.

Pharmaceutically acceptable salts of repaglinide can be prepared in high purity by using the pure repaglinide substantially free of dimer impurity obtained by the method disclosed herein, by known methods.

Repaglinide obtained by the process disclosed herein preferably contains dimer impurity in an amount of less than about 0.25%, more preferably less than 0.15%, still more preferably less than 0.05% and most preferably less than 0.02%.

The total purity of the repaglinide obtained by the process disclosed herein is of greater than about 99.9%, specifically greater than about 99.95%, and more specifically greater than about 99.99% as measured by HPLC.

The term ‘crude repaglinide’ in the specification refers to repaglinide containing the dimer impurity in an amount of greater than about 0.25%.

According to another aspect of the present invention, there is provided a novel compound 2-ethoxy-N-[3-methyl-1-[2-(1-piperidinyl)phenyl]butyl]-4-[2-[[3-methyl-1-[2-(1-piperidinyl)phenyl]butyl]amino]-2-oxoethyl]benzamide, denominated dimer compound II, having the following structural formula:

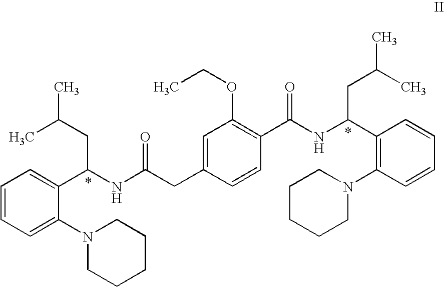

The dimer compound II contains two chiral centers (the asterisk designates the chiral centers) and therefore can exist as four optical isomers. The absolute configuration of these centers may be indicated by the stereochemical descriptors R and S, this R and S notation corresponding to the rules described in Pure Appl. Chem. 1976, 45, 11-30. Unless otherwise mentioned or indicated, the chemical designation of compounds denotes the mixture of all possible stereochemically isomeric forms, said mixtures containing all diastereomers and enantiomers of the basic molecular structure. Stereochemically isomeric forms of the compounds of formula II are obviously intended to be embraced within the scope of the invention.

According to another aspect of the present invention, there is provided an impurity of repaglinide, 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide, designated dimer impurity, having the following structural formula IIa:

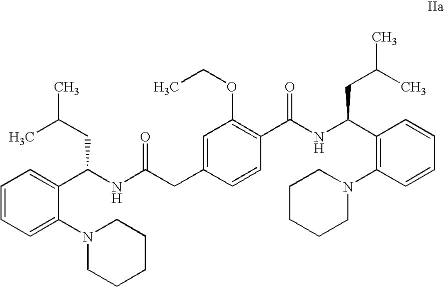

The dimer impurity has the following ¹H NMR (500 MHz, DMSO-d6) d (ppm): 0.89-0.97 (d, 4×3H), 1.3-1.7 (m, 20H), 1.38-1.41 (t, 3H), 2.58-2.6 (m, 2H), 3.08-3.1 (m, 4H), 3.48 (s, 2H), 4.07-4.12 (q, 2H), 5.37-5.38 (m, 1H), 5.5-5.57 (m, 1H), 6.8-7.6 (m, 8H), 6.8-7.6 (d, 3H), 8.32-8.34 (d, 1H), 8.41-8.44 (d, 1H); and MS: m/z: 681.

According to another aspect of the present invention, a process for the preparation of dimer impurity of formula IIa is provided, which comprises:

reacting (S)-3-methyl-1-(2-piperidinophenyl)-1-butylamine of formula III:

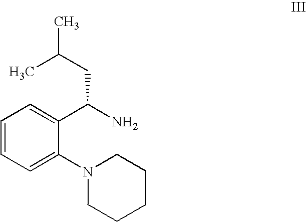

or a salt thereof with 3-ethoxy-4-ethoxycarbonyl phenyl acetic acid of formula VI:

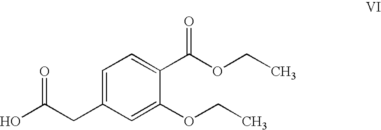

or a salt thereof in the presence of a dehydrating agent selected from boric acid or boric acid derivatives in a suitable solvent, to provide 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide of formula IIa.

Exemplary boric acid derivatives include, but are not limited to, aryl or substituted aryl boronic acids such as phenylboronic acid, 2-chlorophenylboronic acid, 2-nitrophenyl boronic acid, 3-nitrophenylboronic acid, 4-nitrophenylboronic acid, 2-carboxyphenyl boronic acid, 2-chloro-4-carboxyphenylboronic acid, 2-chloro-5-carboxyphenylboronic acid, 3-chloro-4-carboxyphenylboronic acid, 2-chloro-4-fluorophenylboronic acid, 4-chloro-2-fluorophenylboronic acid, 2-chloro-4-methylphenylboronic acid, 2-chloro-5-methylphenylboronic acid, 2-chloro-3-methylpyridine-5-boronic acid, naphthyl boronic acid, and combinations comprising one or more of the foregoing boric acid derivatives. Specific dehydrating agents are boric acid, phenylboronic acid, and combinations comprising one or more of the foregoing dehydrating agents.

Exemplary solvents include, but are not limited to, hydrocarbons, ketones, cyclic ethers, aliphatic ethers, nitriles, alkanes, and the like, and mixtures thereof. Exemplary hydrocarbon solvents include, but are not limited to, toluene, benzene, xylene, and mixtures thereof. Exemplary ketone solvents include, but are not limited to, acetone, methyl isobutyl ketone, and the like, and mixtures thereof. Exemplary cyclic ether solvents include, but are not limited to, tetrahydrofuran, dioxane, and the like, and mixtures thereof. Exemplary nitrile solvents include, but are not limited to, acetonitrile, and the like, and mixtures thereof. Exemplary alkane solvents include, but are not limited to, n-hexane, heptane, cyclohexane, and the like, and mixtures thereof. Specific solvents are toluene, methylene chloride, tetrahydrofuran, acetonitrile, dimethylformamide, and mixtures thereof, and more specifically toluene.

The reaction is carried out at a temperature of −25° C. to the reflux temperature of the solvent used, specifically at a temperature of 0° C. to the reflux temperature of the solvent used, more specifically at a temperature of 25° C. to the reflux temperature of the solvent used, and most specifically at the reflux temperature of the solvent used.

As used herein, “reflux temperature” means the temperature at which the solvent or solvent system refluxes or boils at atmospheric pressure.

The molar ratio of (S)-3-methyl-1-(2-piperidinophenyl)-1-butylamine with respect to 3-ethoxy-4-ethoxycarbonyl phenyl acetic acid is critical in order to ensure a proper course of the reaction. Specifically, about 1 to 5 moles, more specifically, about 1.2 to 3 moles of (S)-3-methyl-1-(2-piperidinophenyl)-1-butylamine is used per 1 mole of 3-ethoxy-4-ethoxycarbonyl phenyl acetic acid.

In an embodiment, the dehydrating agent used in the molar ratio of about 0.01 to 0.5 moles, specifically about 0.01 to 0.2 moles, per 1 mole of (S)-3-methyl-1-(2-piperidinophenyl)-1-butylamine in order to ensure a proper course of the reaction.

In one embodiment, the compound of formula IIa obtained is isolated as solid from a suitable organic solvent by methods usually known in the art such as cooling, partial removal of the solvent from the solution, addition of precipitating solvent, or a combination thereof. Suitable solvents include, but are not limited to, alcohols, hydrocarbons, ketones, cyclic ethers, aliphatic ethers, nitriles, alkanes, and the like, and mixtures thereof.

According to another aspect of the present invention, there is provided a pharmaceutical composition comprising pure repaglinide containing less than about 0.25% of the dimer impurity, or a pharmaceutically acceptable salt thereof, of the present invention and one or more pharmaceutically acceptable excipients.

The present invention further encompasses the use of pure repaglinide containing less than about 0.25% of the dimer impurity, or a pharmaceutically acceptable salt thereof, of the present invention for the manufacture of a pharmaceutical composition.

The repaglinide containing less than about 0.25% of the dimer impurity, or a pharmaceutically acceptable salt thereof, obtained is provided in relatively high purity and also having a relatively low content of one or more organic volatile impurities.

According to another aspect of the present invention, there is provided solid particles of pure repaglinide or a pharmaceutically acceptable salt thereof containing less than about 0.25% of the dimer impurity, wherein 90 volume-% of the particles (D₉₀) have a size of less than or equal to about 400 microns, specifically less than or equal to about 300 microns, more specifically less than or equal to about 100 microns, still more specifically less than or equal to about 60 microns and most specifically less than or equal to about 15 microns.

According to another aspect of the present invention, the particle sizes of pure repaglinide or a pharmaceutically acceptable salt thereof containing less than about 0.25% of the dimer impurity, can be achieved via comminution, or a mechanical process of reducing the size of particles which includes any one or more of cutting, chipping, crushing, milling, grinding, micronizing, trituration or other particle size reduction methods known in the art, to bring the solid state forms the desired particle size range.

The micronized particles of the pure repaglinide containing less than about 0.25% of the dimer impurity, or a pharmaceutically acceptable salt thereof, of present invention may then be formulated into a pharmaceutical composition or dosage form. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. Pure repaglinide containing less than about 0.25% of the dimer impurity, or a pharmaceutically acceptable salt thereof, of the present invention also may be administered as suppositories. The dosage forms may contain the pure repaglinide containing less than about 0.25% of the dimer impurity, or a pharmaceutically acceptable salt thereof, of the present invention as is or, alternatively, as part of a composition. The pharmaceutical compositions may further contain one or more pharmaceutically acceptable excipients as described herein.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants such as magnesium stearate, calcium stearate and sodium stearyl fumarate; flavorings; sweeteners; preservatives; pharmaceutically acceptable dyes and glidants such as silicon dioxide.

Actual dosage levels of pure repaglinide containing less than about 0.25% of the dimer impurity, or a pharmaceutically acceptable salt thereof, of the present invention in the compositions of the invention may be varied to obtain an amount of pure repaglinide containing less than about 0.25% of the dimer impurity, or a pharmaceutically acceptable salt thereof, that is effective to obtain a desired therapeutic response for a particular composition and method of administration.

Provided also herein is an improved process for the preparation of (S)-3-methyl-1-(2-piperidinophenyl)-1-butylamine of formula III:

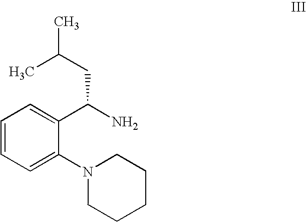

or a salt thereof, which comprises:
a) reacting racemic (±)-3-methyl-1-(2-piperidinophenyl)-1-butylamine of formula IV:

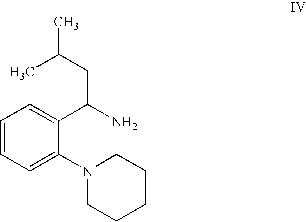

with an optically active di-p-toluoyl-tartaric acid, optionally in the presence of a suitable acid, to produce a diastereomeric excess of di-p-toluoyl-tartaric acid salt compound of formula V:

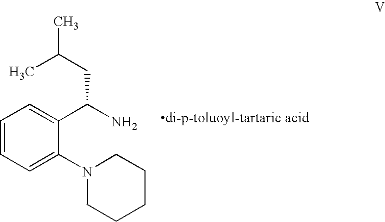

b) if required, separating the diastereomers of formula V; and
c) neutralizing the product of step-(a) or separated diastereomers of step-(b) with a base in a suitable solvent to provide enantiomerically pure compound of formula III.

The term “enantiomerically pure compound of formula III” refers to the compound of formula III having enantiomeric purity greater than about 98%, specifically greater than about 99.9%, more specifically greater than about 99.95%, and most specifically greater than about 99.98% measured by HPLC.

The optically active di-p-toluoyl-tartaric acid used in step-(a) is selected from the group comprising di-p-toluoyl-D-tartaric acid, di-p-toluoyl-L-tartaric acid and hydrates thereof. More preferable optically active acid is di-p-toluoyl-D-tartaric acid.

The optically active di-p-toluoyl-tartaric acid in step-(a) can be optionally used as a mixture with other acids (adjuvant acids) that can be organic or inorganic, such as hydrochloric acid, p-toluenesulphonic acid, methanosulphonic acid or a mixture thereof, in molar proportions that vary between 0.5% and 50% (this molar percentage refers to the total of the mixture of the chiral acid and the adjuvant acid). Most preferable adjuvant acid is p-toluensulphonic acid.

The reaction in step-(a) is carried out in an appropriate solvent or a mixture of appropriate solvents. Appropriate solvents include water, acetone, acetonitrile, methanol, ethanol, isopropanol, tert-butanol, dichloromethane, chloroform, carbon tetrachloride, dimethylformamide, dimethylsulphoxide, ethyl acetate, toluene, xylene, pentane, hexane, heptane, ethyl ether, isopropyl ether, tetrahydrofuran, 1,4-dioxane, ethyleneglycol, 1,2-dimethoxyethane, and in general, any solvent susceptible to being used in a chemical process. Preferable solvents are methanol, ethanol, isopropanol, ethyl acetate, water and mixtures thereof.

The reaction in step-(a) is carried out at a temperature of −20° C. to the reflux temperature of the solvent used, specifically at a temperature of 0° C. to the reflux temperature of the solvent used, and more specifically at a temperature of 20° C. to the reflux temperature of the solvent used.

The term “diastereomeric excess” refers to formation of a diastereomer having one configuration at chiral carbon of formula V in excess over that having the opposite configuration. Preferably, one diastereomer is formed in above about 60% of the mixture of diastereomers over the other, and more preferably above about 80% of the mixture of diastereomers.

The compounds of formula V formed may be used directly in the next step or the compounds of formula V may be isolated from the reaction medium and then used in the next step.

The separation of diastereomers in step-(b) may be required to obtain stereomers with desired optical purity. It is well known that diastereomers differ in their properties such as solubility and then can be separated based on the differences in their properties. The separation of the diastereomers can be performed using the methods known to the person skilled in the art. These methods include chromatographic techniques and fractional crystallization, preferable method being fractional crystallization.

Preferably, a solution of the diastereomeric mixture is subjected to fractional crystallization. The solution of the diastereomeric mixture may be a solution of the reaction mixture obtained as above or a solution prepared by dissolving the isolated diastereomeric mixture in a solvent. Preferable solvents used for the separation include, but are not limited to, water; alcohols such as methanol, ethanol, isopropyl alcohol, propanol, tert-butyl alcohol, n-butanol; ketones such as acetone, methyl ethyl ketone, methyl isobutyl ketone, diethyl ketone; esters such as ethyl acetate, methyl acetate, isopropyl acetate, tert-butyl methyl acetate and ethyl formate; acetonitrile; tetrahydrofuran; dimethylformamide; dimethylsulfoxide; dioxane; diethyl carbonate; and mixtures thereof. Preferable solvents are water, methanol, ethanol, isopropyl alcohol, and mixtures thereof. More preferable solvents are water, methanol, and mixtures thereof.

Fractional crystallization of preferentially one diastereomer from the solution of mixture of diastereomers can be performed by conventional methods such as cooling, partial removal of solvents, using anti-solvent, seeding or a combination thereof.

Fractional crystallization can be repeated until the desired chiral purity is obtained. But, usually one or two crystallizations may be sufficient.

The base used in step-(c) can be an organic or inorganic base. Specific organic bases are triethyl amine, dimethyl amine and tert-butyl amine. Preferable base is an inorganic base. Exemplary inorganic bases include, but are not limited to, hydroxides, carbonates and bicarbonates of alkali or alkaline earth metals. Specific alkali metals are lithium, sodium and potassium, and more specifically sodium and potassium. Specific alkaline earth metals are calcium and magnesium, and more specifically magnesium.

Specific inorganic bases are sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

Exemplary solvents for step-(c) include, but are not limited to, water, alcohols, ketones, cyclic ethers, aliphatic ethers, hydrocarbons, chlorinated hydrocarbons, nitriles, esters and the like, and mixtures thereof. Specific solvents are water, hydrocarbons, alcohols, and mixtures thereof.

Exemplary alcohol solvents include, but are not limited to, C₁ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof, and most specific alcohol solvent is isopropyl alcohol. Exemplary ketone solvents include, but are not limited to, acetone, methyl isobutyl ketone, and the like, and mixtures thereof. Exemplary cyclic ether solvents include, but are not limited to, tetrahydrofuran, dioxane, and the like, and mixtures thereof. Exemplary nitrile solvents include, but are not limited to, acetonitrile and the like, and mixtures thereof. Exemplary ester solvents include, but are not limited to, ethyl acetate, isopropyl acetate, and the like and mixtures thereof. Exemplary hydrocarbon solvents include, but are not limited to, n-pentane, n-hexane and n-heptane and isomers or mixtures thereof, cyclohexane, toluene and xylene. Specific hydrocarbon solvent is toluene. Exemplary chlorinated hydrocarbon solvents include, but are not limited to, methylene chloride, ethyl dichloride, chloroform and carbon tetrachloride or mixtures thereof. Specific chlorinated hydrocarbon solvent is methylene chloride.

Preferable solvent for step-(c) is selected from the group consisting of water, n-pentane, n-hexane, n-heptane, cyclohexane, toluene, xylene, and mixtures thereof.

The enantiomerically pure compound of formula III obtained in step-(c) can be recovered by filtration or centrifugation.

In an embodiment, the resolution procedure of the present invention can be used to resolve mixtures that comprise both enantiomers of the compound of formula IV in any proportion. Therefore, this procedure is applicable both to performing the optical resolution of a racemic mixture of the compound of formula IV (that is to say, that in which the two enantiomers are present in a 1:1 ratio) and for the optical resolution of non-racemic mixtures of the compound of formula IV (in which one of the enantiomers is present in greater proportion), obtained by any physical or chemical method.

The enantiomeric purity of (S)-3-methyl-1-(2-piperidinophenyl)-1-butylamine obtained by the process disclosed herein is of greater than about 98%, specifically greater than about 99.9%, more specifically greater than about 99.95%, and most specifically greater than about 99.98% measured by HPLC.

Repaglinide and pharmaceutically acceptable salts of Repaglinide can be prepared in high purity by using the enantiomerically pure (S)-3-methyl-1-(2-piperidinophenyl)-1-butylamine or its acid addition salts obtained by the methods disclosed herein, by known methods.

Aptly the processes of this invention are adapted to the preparation of repaglinide or a pharmaceutically acceptable salt thereof in high enantiomeric and chemical purity.

According to another aspect of the present invention, there is provided a process for the preparation of highly pure repaglinide or a pharmaceutically acceptable salt thereof substantially free of dimer impurity, comprising the steps of:
a) reacting racemic (±)-3-methyl-1-(2-piperidinophenyl)-1-butylamine of formula IV:

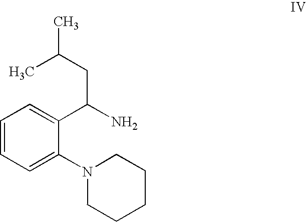

with an optically active di-p-toluoyl-tartaric acid, optionally in the presence of a suitable acid, to produce a diastereomeric excess of di-p-toluoyl-tartaric acid salt compound of formula V:

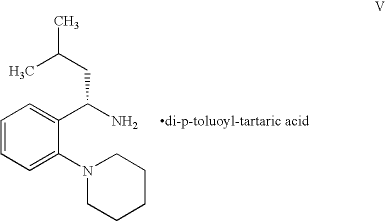

b) if required, separating the diastereomers of formula V; and
c) neutralizing the product of step-(a) or separated diastereomers of step-(b) with a base in a suitable solvent to provide enantiomerically pure (S)-3-methyl-1-(2-piperidinophenyl)-1-butylamine of formula III:

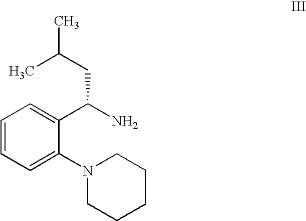

or a salt thereof;
d) reacting the compound of formula III with 3-ethoxy-4-ethoxycarbonyl phenyl acetic acid of formula VI:

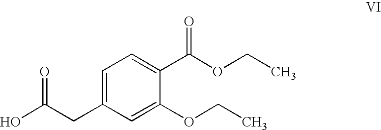

or a salt thereof in the presence of a dehydrating agent in a suitable solvent to produce a compound of formula VII:

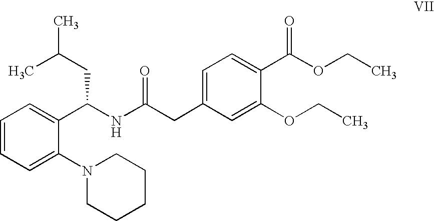

or a salt thereof;
e) deprotecting the compound of formula VII or a salt thereof in the presence of an acid or a base to produce crude repaglinide or a pharmaceutically acceptable salt thereof;
f) providing a solution of crude repaglinide in a solvent selected from the group consisting of aromatic hydrocarbons, esters, polar aprotic solvents, and mixtures thereof;
g) admixing the solution of step-(f) with an anti-solvent selected from the group consisting of hexane, heptane, cyclopentane, cyclohexane, cycloheptane, and mixtures thereof; and
h) recovering pure repaglinide substantially free of dimer impurity, and optionally converting the pure repaglinide obtained into its pharmaceutically acceptable salts thereof.

### HPLC Method for Measuring Chemical Purity:

The purity was measured by HPLC under the following conditions:

| Column | Zorbax SB-Aq (150 × 4.6 mm × 5μ) |
|---|---|
| Mobile phase A | 4.0 gms/Litre solution of potassium dihydrogen |
| | phosphate adjusted to pH 3.20 with dilute H₃PO₄ |
| Mobile phase B | Mobile phase-A, Acetonitrile (300:700 v/v). |
| Diluent | Acetonitrile. |
| Flow rate | 1.5 ml/minute. |
| Run Time | 57.0 min |
| Retention time | 35.346 |

The following examples are given for the purpose of illustrating the present invention and should not be considered as limitation on the scope or spirit of the invention.

### EXAMPLES

### Example 1

### Process for Preparing Pure Repaglinide Substantially Free of Dimer Impurity

Crude repaglinide (15 g, content of dimer impurity: 0.35%) was dissolved in toluene (90 ml) at 60-65° C. This was followed by the addition of cyclohexane (15 ml) to the hot solution at 60-65° C. The solution was then slowly cooled at 25-30° C. and stirred for 1-2 hours. The precipitated product was filtered, washed with cyclohexane (30 ml) and then dried at 50-55° C. under vacuum for 6 hours to produce 12.5 g of pure repaglinide (Yield: 83.3%; Content of dimer impurity by HPLC: 0.06%).

### Example 2

### Preparation of 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide (Dimer Impurity)

In a round bottom flask fitted with a Dean Stark condenser, (S)-3-methyl-1-(2-piperidinophenyl)-1-butylamine (2.0 g, 0.00813 mol) was dissolved in toluene (50 ml), which was followed by the addition of 3-ethoxy-4-ethoxycarbonyl phenyl acetic acid (0.91 g, 0.00406 mol) and phenylboronic acid (0.099 g, 0.000813 mol). The reaction mixture was refluxed for 16-18 hours. The reaction mixture was cooled to 25-30° C. followed by filtration. The toluene layer was washed with water and 1% sodium bicarbonate solution. This was followed by complete distillation of toluene. Hexane (20 ml) was added to the resulting residue in order to precipitate the solid and stirred for 1 hour. The resulting solid was filtered and washed with hexane (10 ml). The crude product was purified by column chromatography to produce 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide (Dimer impurity) with 30% yield.

¹H NMR (500 MHz, DMSO-d6) d (ppm): 0.89-0.97 (d, 4×3H), 1.3-1.7 (m, 20H), 1.38-1.41 (t, 3H), 2.58-2.6 (m, 2H), 3.08-3.1 (m, 4H), 3.48 (s, 2H), 4.07-4.12 (q, 2H), 5.37-5.38 (m, 1H), 5.5-5.57 (m, 1H), 6.8-7.6 (m, 8H), 6.8-7.6 (d, 3H), 8.32-8.34 (d, 1H), 8.41-8.44 (d, 1H); and MS: m/z: 681.

### Example 3

### Preparation of (S)-3-methyl-1-(2-piperidinophenyl)-1-butylamine

### Step-I: Preparation of (S)-3-methyl-1-(2-piperidinophenyl)-1-butylamine di-p-toluoyl-D-tartaric acid salt

Di-p-toluoyl-D-tartaric acid (anhydrous) (1.5 g, 0.004 moles) and p-toluene sulfonic acid (0.77 g, 0.0047 moles) were added to a solution of racemic (±)-3-methyl-1-(2-piperidino-phenyl)-1-butylamine (2.0 g, 0.008 moles) in methanol (15 ml) at 25-30° C. This was followed by the addition of water (5 ml) and the resulting mixture was stirred for 4-5 hours at 25-30° C. The resulted solid was filtered and washed with a mixture of methanol and water (4:1, 10 ml) and dried for 4-6 hours at 50-55° C. to produce 1.5 g of (S)-3-methyl-1-(2-piperidinophenyl)-1-butylamine di-p-toluoyl-D-tartaric acid salt.

### Step-II: Preparation of (S)-3-methyl-1-(2-piperidinophenyl)-1-butylamine

The salt (obtained in step-I) was suspended in a mixture of cyclohexane (15 ml) and water (15 ml). The reaction mixture was basified using a solution of potassium carbonate (0.81 g) in water (15 ml) to adjust pH 9.5-10 at 25-30° C. The organic layer was separated and washed with 10% sodium chloride solution (15 ml). The solvent was evaporated under vacuum below 50° C. to produce 0.5 g of (S)-3-methyl-1-(2-piperidinophenyl)-1-butylamine (Chiral purity by HPLC: 98.4%).

### Example 4

### Preparation of (S)-3-methyl-1-(2-piperidinophenyl)-1-butylamine

### Step-I: Preparation of (S)-3-methyl-1-(2-piperidinophenyl)-1-butylamine di-p-toluoyl-D-tartaric acid salt

Racemic (±)-3-methyl-1-(2-piperidino-phenyl)-1-butylamine (4.0 g, 0.0162 moles) and ethyl acetate (40 ml) were taken in round bottom flask. This was followed by the addition of di-p-toluoyl-D-tartaric acid (5.65, 0.0146 moles) and hexane (16 ml). The resulting mass was stirred for 3-4 hours at 25-30° C. The precipitated solid was filtered and washed with (1:1) mixture of methanol and water (25 ml), and then dried at 50-55° C. for 4-6 hours to give 3.1 g of (S)-3-methyl-1-(2-piperidinophenyl)-1-butylamine di-p-toluoyl-D-tartaric acid salt.

### Step-II: Preparation of (S)-3-methyl-1-(2-piperidinophenyl)-1-butylamine

The salt (obtained in step-I) was suspended in a mixture of cyclohexane (31 ml) and water (31 ml). The reaction mixture was basified using a solution of potassium carbonate (1.7 g) in water (31 ml) to adjust pH 9.5-10 at 25-30° C. The organic layer was separated and washed with 10% sodium chloride solution (31 ml). The solvent was evaporated under vacuum below 50° C. to give 1.0 g of (S)-3-methyl-1-(2-piperidinophenyl)-1-butylamine (Chiral purity by HPLC: 97.5%).

### Example 5

### Preparation of (S)-3-methyl-1-(2-piperidinophenyl)-1-butylamine

### Step-I: Preparation of (S)-3-methyl-1-(2-piperidinophenyl)-1-butylamine di-p-toluoyl-D-tartaric acid salt

Racemic (±)-3-methyl-1-(2-piperidino-phenyl)-1-butylamine (4.0 g, 0.0162 moles) and ethyl acetate (40 ml) were taken in round bottom flask. This was followed by the addition of di-p-toluoyl-D-tartaric acid (3.14 g, 0.008 moles) and p-toluene sulphonic acid (1.4 g, 0.008 moles) in one portion. Hexane (16 ml) was added to the resulting mixture and then stirred for 3-4 hours at 25-30° C. The precipitated solid was filtered and washed with a mixture of methanol and water (1:1, 25 ml). The resulting solid was further dried at 50-55° C. for 4-6 hours to give 3.3 g of (S)-3-methyl-1-(2-piperidinophenyl)-1-butylamine di-p-toluoyl-D-tartaric acid salt.

### Step-II: Preparation of (S)-3-methyl-1-(2-piperidinophenyl)-1-butylamine

The salt (obtained in step-I) was suspended in a mixture of cyclohexane (33 ml) and water (33 ml). The reaction mixture was basified using a solution of potassium carbonate (1.8 g) in water (33 ml) to adjust pH 9.5-10 at 25-30° C. The organic layer was separated and washed with 10% sodium chloride solution (33 ml). The solvent was evaporated under vacuum below 50° C. to give 1.1 g of (S)-3-methyl-1-(2-piperidinophenyl)-1-butylamine (Chiral purity by HPLC: 98.1%).

### Example 6

### Preparation of pure (S)-3-methyl-1-(2-piperidinophenyl)-1-butylamine

### Step-I: Purification of (S)-3-methyl-1-(2-piperidinophenyl)-1-butylamine di-p-toluoyl-D-tartaric acid salt to improve chiral purity

(S)-3-methyl-1-(2-piperidinophenyl)-1-butylamine di-p-toluoyl-D-tartaric acid salt (70 g) was taken in a mixture of methanol (420 ml) and water (280 ml), and the resulting mixture was heated at 55-60° C. for 1 hour. The reaction mass was cooled to 25-30° C. and stirred for 2 hours. The precipitated solid was filtered and washed with (1:1) methanol water mixture (140 ml) and then dried at 50-55° C. for 3-4 hours to give 60 g of (S)-3-methyl-1-(2-piperidinophenyl)-1-butylamine di-p-toluoyl-D-tartaric acid salt (Yield: 85.7%).

### Step-II: Preparation of (S)-3-methyl-1-(2-piperidinophenyl)-1-butylamine

The salt (obtained in step-I) was suspended in a mixture of cyclohexane (600 ml) and water (600 ml). The reaction mixture was basified using a solution of potassium carbonate (32.7 g) in water (600 ml) to adjust pH 9.5-10 at 25-30° C. The organic layer was separated and washed with 10% sodium chloride solution (600 ml). The solvent was evaporated under vacuum below 50° C. to give 21 g of (S)-3-methyl-1-(2-piperidinophenyl)-1-butylamine (Yield: 90%; Chiral purity by HPLC: 99.94%).

### Example 7

### Preparation of pure (S)-3-methyl-1-(2-piperidinophenyl)-1-butylamine

### Step-I: Preparation of (S)-3-methyl-1-(2-piperidinophenyl)-1-butylamine di-p-toluoyl-D-tartaric acid salt

Di-p-toluoyl-D-tartaric acid (613 g, 1.58 moles) was added to a solution of racemic (±)-3-methyl-1-(2-piperidino-phenyl)-1-butylamine (600.0 g, 1.58 moles) in methanol (4.2 L) at 25-30° C. and stirred for 3-4 hours. The resulting solid was cooled to 5-10° C., filtered and washed with a mixture of methanol and water (1:1, 1.2 L) and dried at 50-55° C. for 4-6 hours to obtain 442 g of (S)-3-methyl-1-(2-piperidinophenyl)-1-butylamine di-p-toluoyl-D-tartaric acid salt.

### Step-II: Preparation of pure (S)-3-methyl-1-(2-piperidinophenyl)-1-butylamine

The salt (obtained in step-I) was suspended in a mixture of cyclohexane (4.4 L) and water (4.4 L). The reaction mixture was basified using a solution of potassium carbonate (241 g) in water (4.4 L) to adjust pH 9.5-10 at 25-30° C. The organic layer was separated and washed with 10% sodium chloride solution (4.4 L). The solvent was evaporated under vacuum below 50° C. to give 154.8 g of (S)-3-methyl-1-(2-piperidinophenyl)-1-butylamine (Chiral purity by HPLC: 99.98%).

### Example 8

### Preparation of Ethyl (S)-2-ethoxy-4-[N-[1-(2-piperidinophenyl)-3-methyl-1-butyl]aminocarbonylmethyl]benzoate

In a round bottom flask fitted with a Dean Stark condenser, 3-ethoxy-4-ethoxycarbonyl phenyl acetic acid (10.26 g, 0.0426 moles) was dissolved in toluene (100 ml) followed by slow addition of phenylboronic acid (0.494 g, 0.0040 moles) and (S)-3-methyl-1-(2-piperidinophenyl)-1-butylamine (10 g, 0.0406 moles). The reaction mixture was refluxed for 16-18 hours. The reaction mixture was cooled at room temperature followed by filtration. The toluene layer was washed with water and 1% sodium bicarbonate solution. This was followed by complete distillation of toluene. Hexane (50 ml) was added to the resulting residue in order to precipitate the solid and then stirred for 1 hour. The resulting solid was filtered and washed with hexane (10 ml). The wet material was further dried at 50-55° C. under vacuum for 4-6 hours to produce ethyl (S)-2-ethoxy-4-[N-[1-(2-piperidinophenyl)-3-methyl-1-butyl]aminocarbonylmethyl]benzoate (Yield: 89.6%; HPLC purity: 99.46%; and Chiral purity: 99.98%).

### Example 9

### Process for the preparation of 2-ethoxy-4-[2-[[(1S)-3-methyl-1-[2-(1-piperidinyl)phenyl]butyl]amino]-2-oxoethyl]benzoic acid (Repaglinide)

Ethyl(S)-2-ethoxy-4-[N-[1-(2-piperidinophenyl)-3-methyl-1-butyl]aminocarbonyl methyl]benzoate (4.5 g, 0.0093 moles) was dissolved in methanol (45 ml), which was followed by the addition of sodium hydroxide solution (0.75 g of sodium hydroxide dissolved in 6 ml water). The reaction mixture was heated at 60-65° C. for 3-4 hours. Methanol (80-85%) was removed from the reaction mixture under vacuum. The remaining reaction mixture was diluted with water (45 ml) and pH was adjusted to 6.5-7.0 with 1N HCl. The precipitated solid was stirred for 2-3 hours followed by filtration and washing with water (45 ml). The product was further dried at 50-55° C. under vacuum for 6-8 hours to produce repaglinide (Yield: 83.2%; HPLC purity: 99.81%; and Chiral purity by HPLC: 99.97%).

1. (canceled)

2. (canceled)

3. (canceled)

4. (canceled)

5. A process for the preparation of enantiomerically pure (S)-3-methyl-1-(2-piperidinophenyl)-1-butylamine of formula III: 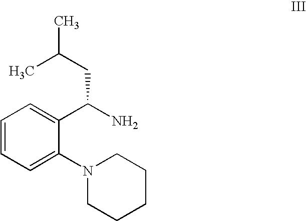
or a salt thereof, comprising the steps of;
a) reacting racemic (+)-3-methyl-1-(2-piperidinophenyl)-1-butylamine of formula IV: 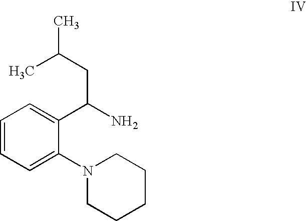
with an optically active di-p-toluoyl-tartaric acid, optionally in the presence of a suitable acid (adjuvant acid) selected from the group consisting of organic and inorganic acids, to produce a diastereomeric excess of di-p-toluoyl-tartaric acid salt compound of formula V: 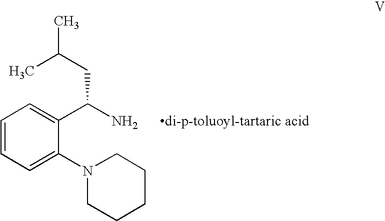
b) if required, separating the diastereomers of formula V;
c) neutralizing the product of step-(a) or separated diastereomers of step-(b) with a base in a suitable solvent; and
d) recovering enantiomerically pure (S)-3-methyl-1-(2-piperidinophenyl)-1-butylamine of formula III, and optionally converting the product obtained into its salts thereof;
wherein the optically active di-p-toluoyl-tartaric acid used in step-(a) is selected from the group consisting of di-p-toluoyl-D-tartaric acid, di-p-toluoyl-L-tartaric acid and hydrates thereof.

6. (canceled)

7. The process of claim 5, wherein the optically active chiral acid is di-p-toluoyl-D-tartaric acid.

8. (canceled)

9. The process of claim 5, wherein the adjuvant acid used in step-(a) is selected from the group consisting of hydrochloric acid, p-toluenesulphonic acid, methanosulphonic acid, and mixtures thereof.

10. (canceled)

11. The process of claim 5, wherein the reaction in step-(a) is carried out in a solvent selected from the group consisting of water, acetone, acetonitrile, methanol, ethanol, isopropanol, tert-butanol, dichloromethane, chloroform, carbon tetrachloride, dimethylformamide, dimethylsulphoxide, ethyl acetate, toluene, xylene, pentane, hexane, heptane, ethyl ether, isopropyl ether, tetrahydrofuran, 1,4-dioxane, ethyleneglycol, 1,2-dimethoxyethane, and mixtures thereof; wherein the separation in step-(b) is carried out in a solvent selected from the group consisting of water, alcohols, ketones, esters, acetonitrile, tetrahydrofuran, dimethylformamide, dimethylsulfoxide, dioxane, diethyl carbonate, and mixtures thereof; and wherein the solvent used in step-(c) is selected from the group consisting of water, alcohols, ketones, cyclic ethers, aliphatic ethers, hydrocarbons, chlorinated hydrocarbons, nitriles, esters, and mixtures thereof.

12. The process of claim 11, wherein the solvent used in step-(a) is selected from the group consisting of methanol, ethanol, isopropanol, ethyl acetate, water, and mixtures thereof; wherein the solvent used in step-(b) is selected from the group consisting of water, methanol, ethanol, isopropyl alcohol, and mixtures thereof; and wherein the solvent used in step-(c) is selected from the group consisting of water, n-pentane, n-hexane, n-heptane, cyclohexane, toluene, xylene, and mixtures thereof.

13. The process of claim 5, wherein the reaction in step-(a) is carried out at a temperature of −20° C. to the reflux temperature of the solvent used; wherein the separation of the diastereomers in step-(b) is performed by using chromatographic techniques or fractional crystallization; and wherein the enantiomerically pure compound of formula III obtained in step-(d) is recovered by filtration or centrifugation.

14. (canceled)

15. (canceled)

16. The process of claim 13, wherein the separation in step-(b) is carried out by fractional crystallization, and wherein the fractional crystallization is performed by cooling, partial removal of solvents, using anti-solvent, seeding or a combination thereof.

17. (canceled)

18. (canceled)

19. (canceled)

20. The process of claim 5, wherein the base used in step-(c) is an organic or inorganic base; and wherein the inorganic base is selected from the group consisting of sodium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide.

21. (canceled)

22. (canceled)

23. (canceled)

24. (canceled)

25. (canceled)

26. (canceled)

27. The process of claim 5, wherein the (S)-3-methyl-1-(2-piperidinophenyl)-1-butylamine obtained has an enantiomeric purity of about 98% to about 99.98% as measured by HPLC.

28. (canceled)

29. (canceled)

30. (canceled)

31. (canceled)

32. A process for the preparation of highly pure repaglinide or a pharmaceutically acceptable salt thereof, using the (S)-3-methyl-1-(2-piperidinophenyl)-1-butylamine of formula III or a salt thereof as prepared according to the process of claim 5, comprising:
a) reacting the (S)-3-methyl-1-(2-piperidinophenyl)-1-butylamine of formula III or a salt thereof with 3-ethoxy-4-ethoxycarbonylphenyl acetic acid of formula VI: 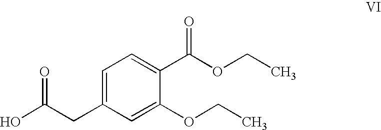
or a salt thereof in the presence of a dehydrating agent in a suitable solvent to produce ethyl (S)-2-ethoxy-4-[N-[1-(2-piperidinophenyl)-3-methyl-1-butyl]amino carbonylmethyl]benzoate of formula VII: 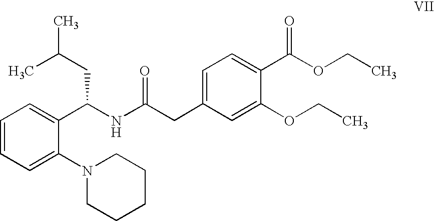
or a salt thereof;
b) deprotecting the compound of formula VII or a salt thereof in the presence of an acid or a base to produce crude repaglinide or a pharmaceutically acceptable salt thereof;
c) providing a solution of crude repaglinide in a solvent selected from the group consisting of aromatic hydrocarbons, esters, polar aprotic solvents, and mixtures thereof;
d) admixing the solution of step-(c) with an anti-solvent; and
e) recovering pure repaglinide substantially free of dimer impurity, and optionally converting the pure repaglinide obtained into its pharmaceutically acceptable salts thereof.

33. The process of claim 32, wherein the solvent used in step-(c) is selected from the group consisting of toluene, xylene, methyl acetate, ethyl acetate, n-propyl acetate, isopropyl acetate, n-butyl acetate, isobutyl acetate, tert-butyl acetate, ethyl formate, N,N-dimethylformamide, N,N-dimethylacetamide, dimethylsulfoxide, and mixtures thereof; and wherein the anti-solvent used in step-(d) is selected from the group consisting of C₃ to C₇ straight or cyclic aliphatic hydrocarbon solvents, and mixtures thereof.

34. The process of claim 33, wherein the solvent used in step-(c) is toluene; and wherein the anti-solvent is selected from the group consisting of hexane, heptane, cyclopentane, cyclohexane, cycloheptane, and mixtures thereof.

35. The process of claim 32, wherein the solution in step-(c) is prepared by dissolving crude repaglinide in a suitable solvent at a temperature of above about 25° C.; and wherein the solution obtained in step-(c) is optionally subjected to carbon treatment.

36. (canceled)

37. (canceled)

38. (canceled)

39. (canceled)

40. (canceled)

41. (canceled)

42. The process of claim 32, wherein the admixing in step-(d) is carried out by adding anti-solvent to the solution or by adding the solution to the anti-solvent; and wherein the addition is carried out at a temperature of about 40° C. to about 80° C. for at least 20 minutes.

43. (canceled)

44. (canceled)

45. The process of claim 32, wherein the recovery of pure repaglinide in step-(e) is performed by filtration or centrifugation.

46. The process of claim 32, wherein the repaglinide obtained has a total purity of about 99.9% to about 99.99% as measured by HPLC.

47. (canceled)

48. Repaglinide or a pharmaceutically acceptable salt thereof, in which repaglinide has a total purity greater than about 99% and further comprises the dimer of formula II: 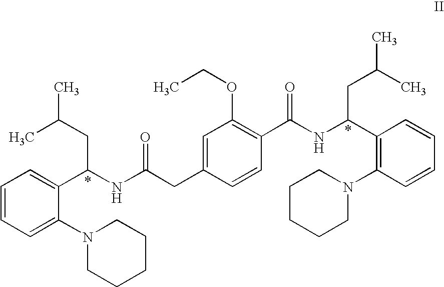
or a stereochemically isomeric form or a mixture of stereochemically isomeric forms thereof, in less than about 0.25% (on a w/w basis).

49. Repaglinide of claim 48, comprising the dimer compound of formula II or a stereochemically isomeric form or a mixture of stereochemically isomeric forms thereof in an amount of about 0.02% to about 0.15%.

50. (canceled)

51. Repaglinide of claim 48, comprising less than about 0.02% of the dimer compound of formula II or a stereochemically isomeric form or a mixture of stereochemically isomeric forms thereof.

52. (canceled)

53. Repaglinide or a pharmaceutically acceptable salt thereof, in which repaglinide has a total purity greater than about 99% and further comprises the dimer of formula IIa: 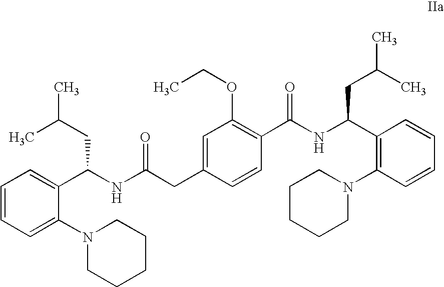
in less than about 0.25% (on a w/w basis).

54. Repaglinide of claim 53, comprising the dimer impurity of formula IIa in an amount of about 0.02% to about 0.15%.

55. (canceled)

56. Repaglinide of claim 53, comprising less than about 0.02% of the dimer impurity of formula IIa.

57. (canceled)

58. Repaglinide of anyone of claims 48-56, having a total purity of about 99% to about 99.99%.

59. (canceled)

60. (canceled)

61. (canceled)

62. A pharmaceutical composition comprising pure repaglinide as in claims 48 to 58 or a pharmaceutically acceptable salt thereof containing less than about 0.25% of the dimer compound of formula II or the dimer compound of formula IIa and one or more pharmaceutically acceptable excipients.

63. (canceled)

64. The pharmaceutical composition of claim 62, wherein the pharmaceutical composition is selected from dosage forms comprising liquid, powder, elixir and injectable solution.

65. (canceled)

66. A pharmaceutical composition comprising crystalline particles of pure repaglinide or a pharmaceutically acceptable salt thereof containing less than about 0.25% of the dimer impurity, wherein 90 volume-% of the particles (D₉₀) have a size of less than or equal to about 400 microns.

67. The pharmaceutical composition of claim 66, wherein the 90 volume-% of the particles (D₉₀) have a size of less than or equal to about 300 microns; less than or equal to about 100 microns; or less than or equal to about 15 microns.

68. (canceled)

69. (canceled)

70. A compound having the formula II, its stereochemically isomeric forms, and a mixture of stereochemically isomeric forms thereof:

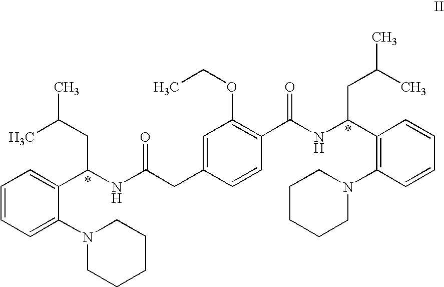

71. A dimer impurity of repaglinide, 2-ethoxy-N-[(1S)-3-methyl-1-[2-(1-piperidinyl)phenyl]butyl]-4-[2-[[(1S)-3-methyl-1-[2-(1-piperidinyl)phenyl]butyl]amino]-2-oxoethyl]benzamide, having the following structural formula IIa:

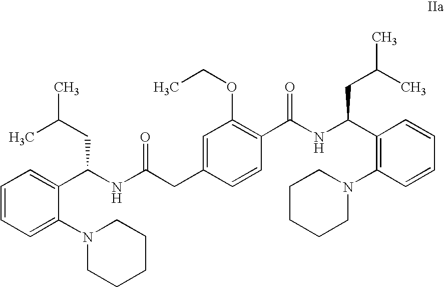

72. A process for preparing the compound of claim 71 comprising:
reacting (S)-3-methyl-1-(2-piperidinophenyl)-1-butylamine of formula III: 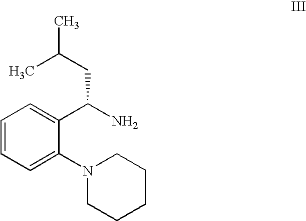
or a salt thereof with 3-ethoxy-4-ethoxycarbonyl phenyl acetic acid of formula VI: 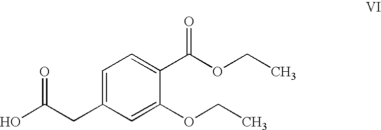
or a salt thereof in the presence of a dehydrating agent and a suitable solvent to produce the compound of formula IIa;
wherein the dehydrating agent is boric acid, a boric acid derivative, or a combination thereof.

73. (canceled)

74. The process of claim 72, wherein the boric acid derivative is an aryl or substituted aryl boronic acid selected from the group consisting of phenylboronic acid, 2-chlorophenylboronic acid, 2-nitrophenyl boronic acid, 3-nitrophenylboronic acid, 4-nitrophenylboronic acid, 2-carboxyphenyl boronic acid, 2-chloro-4-carboxyphenylboronic acid, 2-chloro-5-carboxyphenylboronic acid, 3-chloro-4-carboxyphenylboronic acid, 2-chloro-4-fluorophenylboronic acid, 4-chloro-2-fluorophenylboronic acid, 2-chloro-4-methylphenylboronic acid, 2-chloro-5-methylphenylboronic acid, 2-chloro-3-methylpyridine-5-boronic acid, naphthyl boronic acid, and combinations comprising one or more of the foregoing boric acid derivatives.

75. A process for purifying repaglinide, comprising:
a) providing a solution of crude repaglinide in a solvent selected from the group consisting of aromatic hydrocarbons, esters, polar aprotic solvents, and mixtures thereof
b) admixing the solution of step-(a) with an anti-solvent selected from the group consisting of C₃ to C₇ straight or cyclic aliphatic hydrocarbon solvents, and mixtures thereof and
c) recovering pure repaglinide substantially free of dimer impurity.

76. (canceled)

77. The process of claim 75, wherein the solvent used in step-(a) is selected from the group consisting of toluene, xylene, methyl acetate, ethyl acetate, n-propyl acetate, isopropyl acetate, n-butyl acetate, isobutyl acetate, tert-butyl acetate, ethyl formate, N,N-dimethylformamide, N,N-dimethylacetamide, dimethylsulfoxide, and mixtures thereof; and wherein the anti-solvent used in step-(b) is selected from the group consisting of hexane, heptane, cyclopentane, cyclohexane, cycloheptane, and mixtures thereof.

78. The process of claim 77, wherein the solvent used in step-(a) is toluene, and wherein the anti-solvent is cyclohexane.

79. The process of claim 75, wherein the solution in step-(a) is prepared by dissolving crude repaglinide in a suitable solvent at a temperature of above about 25° C.; and wherein the solution obtained in step-(a) is optionally subjected to carbon treatment.

80. (canceled)

81. (canceled)

82. (canceled)

83. (canceled)

84. (canceled)

85. (canceled)

86. The process of claim 75, wherein the admixing in step-(b) is carried out by adding anti-solvent to the solution or by adding the solution to the anti-solvent; and wherein the addition is carried out at a temperature of about 40° C. to about 80° C.

87. (canceled)

88. (canceled)

89. The process of claim 75, wherein the recovery of pure repaglinide substantially free of dimer impurity in step-(c) is performed by filtration or centrifugation.

90. The process of claim 75, wherein the repaglinide obtained has dimer impurity in an amount of about 0.02% to about 0.25%.

91. (canceled)

92. (canceled)

93. The process of claim 75, wherein the repaglinide obtained has dimer impurity in an amount of less than about 0.02%.

94. The process of claim 75, wherein the repaglinide obtained has a total purity of about 99.9% to about 99.99%.

95. (canceled)

96. (canceled)
